(19) United States
(12) Patent Application Publication
(10) Pub. No.: US 2023/0381381 A1
(43) Pub. Date: Nov. 30, 2023

(54) FLEXIBLE TISSUE REPAIR PATCH

(71) Applicant: UNIVERSITY HOSPITALS CLEVELAND MEDICAL CENTER, Cleveland, OH (US)

(72) Inventors: Rachel Pope, Cleveland, OH (US); Ozan Akkus, Cleveland, OH (US); Adonis Hijaz, Cleveland, OH (US); Shubham Gupta, Cleveland, OH (US); Ilaha Isali, Cleveland, OH (US)

(21) Appl. No.: 18/307,287

(22) Filed: Apr. 26, 2023

Related U.S. Application Data
(60) Provisional application No. 63/334,732, filed on Apr. 26, 2022.

(51) Int. Cl. A61L 31/00; A61L 31/16; A61F 2/00; A61L 31/06

(52) U.S. Cl. CPC A61L 31/005; A61L 31/16; A61F 2/0063; A61L 2300/414; A61F 2210/0076; A61L 2430/22; A61L 31/06

(57) ABSTRACT

A tissue repair patch having an outer side and an inner side is described. The tissue repair patch includes a structural component comprising collagen and/or chorion and a regenerative component comprising amniotic tissue. Methods of tissue repair using the tissue repair patch are also described.

### CROSS-REFERENCE TO RELATED APPLICATIONS

This application claims priority to U.S. Provisional Patent Application Ser. No. 63/333,732, filed Apr. 26, 2022, which is incorporated herein by reference.

### BACKGROUND

Replacing bladder tissue with a functional equivalent remains one of the most challenging problems for conditions such as iatrogenic injuries, trauma, or fistula in reconstructive urology. Pokrywczynska et al., Experimental Biology and Medicine, 239(3): 264-271 (2014). Nearly 3.5 million women worldwide currently live with an obstetric-related vesicovaginal (VVF) fistula. Approximately 100,000 new cases develop each year, primarily affecting women in low-resource countries. Development of a durable cell-seeded hybrid scaffold that would not require the extensive dissection or suturing required during standard surgical repair of a VVF fistula could be a potential treatment option for patients with a VVF.

Attempts have been made to develop suitable biomaterials for urological tissue engineering using both natural and synthetic materials, for example polyglycolic acid and poly L-lactic acid, and naturally derived materials including SIS, Permacol™. and porcine bladder matrix. See for example Barski et al., Int. J. Med. Sci. 14(4), 310-318 (2017) and Joseph et al., J. Urol. 191(5), 1389-1395 (2014). However, none of these materials have been found to be totally successful vis a vis immunogenicity and rejection and recellularization. Accordingly, there remains a need for biomaterials suitable for bladder repair.

### SUMMARY OF THE INVENTION

Amnion tissue has been found to be not only regenerative, but also non-allogenic and highly sustainable in the bladder and in the vagina. Utilization of naturally derived biomaterials such as amnion and collagen can provide biochemical cues for cell attachment, growth, and proliferation. Additionally, oral epithelial cells represent a viable source of cells for clinical applications as they can be collected from small biopsies and expanded for tissue regeneration. Development of durable scaffold such as multiphasic balder patch could enhance healing a vesico-vaginal fistula and would not require the extensive dissection or suturing required during standard surgical repair of a vesico-vaginal fistula. It also would be safe if placed near the ureteral orifices and therefore, the inherent risk of injury to the ureters of VVF repair could be avoided.

The inventors have developed a bioengineered regenerative cell-seeded multiphasic bladder patch for bladder reconstruction. Human oral epithelial cells were harvested and expanded from buccal grafts under an IRB-approved protocol. Electrochemically compacted collagen sheets were fabricated using planar electrodes and then crosslinked using genipin (2% genipin in 90% ethanol). The human amniotic membrane was isolated from the placenta and adhered to a genipin crosslinked collagen sheet/PCL using the gluing solution containing sodium hyaluronate, bovine serum albumin and dextran. The morphology and composition of the surface layer were evaluated by scanning electron microscopy (SEM). Human oral epithelial cells were grown in culture and seeded on a scaffold made of the amnion and electrocompacted collagen.

### BRIEF DESCRIPTION OF THE FIGURES

FIG. 1 provides a cut-away side view of the tissue repair patch.

FIG. 2 provides a flow chart of the steps involved in the preparation of electrocompacted amnion and collagen for the tissue repair patch.

FIGS. 3A and 3B provides a schematic depiction of bioengineered scaffold for bladder regeneration, showing A) placement of the scaffold on the bladder and B) the layers making up the scaffold. Cells were seeded on amnion section of bladder patch.

FIGS. 4A, 4B, 4C, and 4D provide graphs and images showing A) representative immunofluorescent images of human oral epithelial cells CK3/2p (green)/DAPI (blue), Scale bar is 100 μm; B) SEM shows adhesion between collagen sheet and amnion; C) MTT proliferation assay of cells seeded on bladder patch, TCP-tissue culture plate; D) Western Blot images of cells were seeded on amnion portion of bladder patch.

### DETAILED DESCRIPTION OF THE INVENTION

The present invention provides a tissue repair patch having an outer side and an inner side is described. The tissue repair patch includes a structural component comprising collagen and/or chorion and a regenerative component comprising amniotic tissue. Methods of tissue repair using the tissue repair patch are also provided.

The terminology as set forth herein is for description of the embodiments only and should not be construed as limiting of the invention as a whole. Unless otherwise specified, “a,” “an,” “the,” and “at least one” are used interchangeably. Furthermore, as used in the description of the invention and the appended claims, the singular forms “a”, “an”, and “the” are inclusive of their plural forms, unless contraindicated by the context surrounding such.

Also herein, the recitations of numerical ranges by endpoints include all numbers subsumed within that range (e.g., 1 to 5 includes 1, 1.5, 2, 2.75, 3, 3.80, 4, 5, etc.).

As used herein, the term “about,” when referring to a value or to an amount of mass, weight, time, volume, concentration or percentage is meant to encompass variations of in some embodiments±20%, in some embodiments±10%, in some embodiments±5%, in some embodiments±1%, in some embodiments±0.5%, and in some embodiments±0.1% from the specified amount, as such variations are appropriate to perform the disclosed method.

The terms “comprises,” “comprising,” “includes,” “including,” “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

The conjunctive phrase “and/or” indicates that either or both of the items referred to can be present.

A “subject,” as used herein, can be any animal, and may also be referred to as the patient. Preferably the subject is a vertebrate animal, and more preferably the subject is a mammal, such as a research animal (e.g., a mouse or rat) or a domesticated farm animal (e.g., cow, horse, pig) or pet (e.g., dog, cat). In some embodiments, the subject is a human.

The terms “therapeutically effective” and “pharmacologically effective” are intended to qualify the amount of each agent which will achieve the goal of decreasing disease severity while avoiding adverse side effects such as those typically associated with alternative therapies. The therapeutically effective amount may be administered in one or more doses.

“Biocompatible” as used herein, refers to any material that does not cause injury or death to a subject or induce an adverse reaction in a subject when placed in contact with the subject's tissues. Adverse reactions include for example inflammation, infection, fibrotic tissue formation, cell death, or thrombosis. The terms “biocompatible” and “biocompatibility” when used herein are art-recognized and mean that the material is neither itself toxic to a subject, nor degrades (if it degrades) at a rate that produces byproducts at toxic concentrations, does not cause prolonged inflammation or irritation, or does not induce more than a basal immune reaction in the host.

As used herein, “treatment” means any manner in which the symptoms of a defect, condition, disorder, or disease, or any other indication, are ameliorated or otherwise beneficially altered.

### Tissue Repair Patch

In one aspect, the present invention provides a tissue repair patch having an outer side and an inner side, comprising a structural component comprising collagen and/or chorion and a regenerative component comprising amniotic tissue. The tissue repair patch provides structural integrity for a tissue wound or tissue defect, while providing a surface that facilitates healing of the wound or defect. The inner side of the tissue repair patch will be positioned over (and typically in contact with) the wound or defect, while the outer side faces away from the tissue upon which the patch has been positioned.

The tissue repair patch includes two main components; a structural component and a regenerative component. The structural component is primarily intended to provide strength and flexibility for the tissue repair patch, while the regenerative component improves the ability of the tissue wound or tissue defect to heal. The structural component comprises collagen and/or chorion. Accordingly, in some embodiments the structural component comprises collagen, and in other embodiments the structural component comprises chorion. In some embodiments, the structural component comprises collagen and chorion. The collagen and chorion can be mixed and/or compacted together; e.g., in some embodiments the structural component comprises electrocompacted collagen and chorion. The chorion and amnion can be decellularized using, for example, a detergent.

In some embodiments, the structural component and the regenerative component are in separate adjacent layers, with the structural component being on the outer side and the regenerative component being on the inner side. In the context, the outer layer refers to the side facing away from the tissue when the tissue repair patch has been placed on a tissue wound or defect, while the inner side refers to the side facing the tissue (or the wound or defect in that tissue). When the structural and regenerative components are provides as separate layers, the two layers can be adhered to one another using crosslinking or an adhesive (e.g., a glue). A wide variety of suitable biocompatible adhesives can be used. In some embodiments, the structural component is adhered to the regenerative component using a glue comprising sodium hyaluronate, dextran, and bovine serum albumin.

In some embodiments, the structural component and the regenerative component are mixed together in a single layer. This can be done by breaking down the two components into small pieces which are then combined to form a single layer. For example, the two tissues can be chopped into small pieces, mixed in an aqueous solution, and then lyophilized to form a layer. In some embodiments, the layer can be electro-compacted to further mix and adhere the two components. When the structural component and the regenerative component are mixed, the ratio of the two components can vary. For example, the ratio of the regenerative component to the structural component can vary from 1:5 to 5:1, from 1:4 to 4:1, from 1:3 to 3:1, from 1:2 to 2:1, from 1.5:1 to 1:1.5, or the two can be present in about a 1:1 ratio.

The structural component of the tissue repair patch comprises collagen and/or chorion tissue. The collagen and/or chorion can be modified to provide the desired level of strength and flexibility. For example, the collagen can be crosslinked collagen (e.g., genepin-crosslinked collagen). The structure component can comprise collagen, chorion, or a mixture of the two. The materials can be normal (i.e., cellular) or decellularized to isolate the extracellular matrix of the tissue from inhabiting cells. Note that where the structural component comprises both collagen and chorion that have been electrocompacted, they can be mixed using a procedure similar to that described for mixing the structural and regenerative components.

In some embodiments, the tissue repair patch further comprises epithelial cells, which are seeded on the inner side of the tissue repair patch. A wide variety of different types of epithelial cells can be used. For example, urogenital epithelial cells, or oral epithelial cells can be used. In some embodiments, the epithelial cells are oral or urogenital epithelial cells. When intended for use with human subjects, the epithelial cells are preferably human epithelial cells.

In some embodiments, the tissue repair patch also includes a biocompatible polymer layer on the outer side of the tissue repair patch. The biocompatible polymer layer is positioned adjacent to the outer side of the structural layer. A wide variety of biocompatible polymers are known to those skilled in the art. Examples of biocompatible polymers include natural or synthetic polymers such as polystyrene, polylactic acid, polyketal, butadiene styrene, styreneacrylic-vinyl terpolymer, polymethylmethacrylate, polyethylmethacrylate, polyalkylcyanoacrylate, styrene-maleic anhydride copolymer, polyvinyl acetate, polyvinylpyridine, polydivinylbenzene, polybutyleneterephthalate, acrylonitrile, vinylchloride-acrylates, polycaprolactone, poly(alkyl cyanoacrylates), poly(lactic-co-glycolic acid), and the like. In some embodiments, the polymer layer comprises polycaprolactone (PCL). In some embodiments, the biocompatible polymer layer can be formed into a mesh (e.g., a hexagonal mesh) to improve the flexibility of the polymer layer.

In some embodiments, the biocompatible polymer is a biodegradable polymer. Examples of biodegradable polymers include polylactide polymers include poly(D,L-Lactide)s; poly(lactide-co-glycolide) (PLGA) copolymers; polyglycolide (PGA) and polydioxanone; caprolactone polymers; chitosan; hydroxybutyric acids; polyanhydrides and polyesters; polyphosphazenes; and polyphosphoesters.

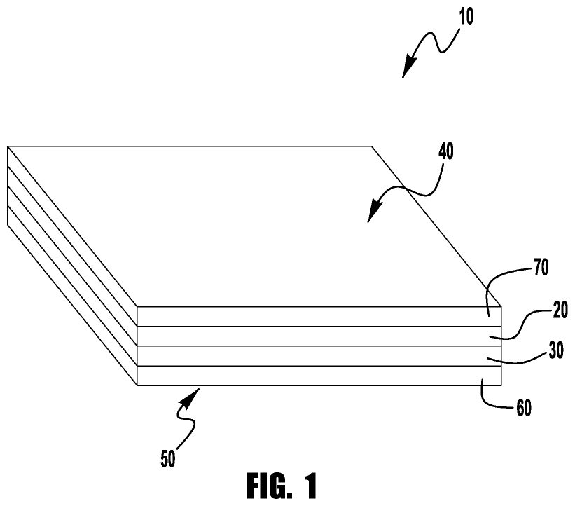

The structure of an embodiment of the tissue repair patch 10 is shown in FIG. 1. The tissue repair patch includes a structural layer 20 adjacent to a regenerative layer 30. The structural layer 20 faces the outer side 40 of the tissue repair patch 10, while the regenerative layer 30 faces the inner side 50 of the tissue repair patch 10. A layer of epithelial cells 60 are seeded adjacent to the regenerative layer 30 on the inner side 50 of the tissue repair patch 10. A biocompatible polymer layer 70 is positioned adjacent to the structural layer 20 on the outer side 40 of the tissue repair patch 10.

The tissue repair patch can be provided with a number of sizes and characteristics. The tissue repair patch can be provided in any suitable shape for the tissue wound or defect, such as square, circular, or rectangular. In addition, the tissue repair patch should have a size somewhat greater than the size of the tissue wound or defect so that the repair patch can cover the wound or defect while providing additional contact with healthy tissue to allow for adhesion to the healthy tissue. For example, in some embodiments, the tissue repair patch has a size ranging from 4 cm² to 100 cm². The tissue repair patch should also have a thickness suitable for the type of tissue where it is being used. In some embodiments, the tissue repair patch has a thickness ranging from 0.1 mm to 3 mm. The tissue repair patch should also have sufficient strength and flexibility to allow it to remain intact at the tissue wound or defect site, while having the flexibility to move as the tissue in that area normally would. Accordingly, in some embodiments, the tissue repair patch has a modulus of elasticity (MPa) ranging from 0.1 MPa to 10 MPa. If treating stiffer tissue such as a tendon or ligament, even higher elasticity values, up to 500 MPa, can be used.

In some embodiments, the tissue repair patch can function as a tissue scaffold. A tissue scaffold is a support structure that provides a matrix for cells to guide the process of wound healing. The morphology of the scaffold guides cell migration and cells are able to migrate into or over the scaffold, respectively. The cells then are able to proliferate and synthesize new tissue.

The tissue repair patch can also include one or more growth factors on the inner and/or outer side of the tissue repair patch. The growth factors used for the inner and outer side of the tissue repair patch will often differ, due to the different functions provided by the outer and inner sides of the tissue repair patch. Examples of suitable growth factors include platelet-derived growth factors, transforming growth factor α, TGFβ1, basic fibroblast growth factor, epidermal growth factor, placental growth factor, and granulocyte colony-stimulating factor.

### Methods of Tissue Repair

Another aspect of the invention provides a method of tissue repair. The method includes the step of positioning a tissue repair patch on a tissue wound or defect of a subject, the tissue repair patch having an outer side and an inner side, comprising a structural component comprising collagen and/or chorion and a regenerative component comprising amniotic tissue.

The tissue repair patch used in the method of tissue repair can have any of the features described herein. For example, in some embodiments the structural component and the regenerative component of the tissue repair patch are in separate adjacent layers, with the structural component being on the outer side and the regenerative component being on the inner side, while in other embodiments the structural component and the regenerative component of the tissue repair patch are mixed together in a single layer. In further embodiments, the tissue repair patch further comprises epithelial cells seeded on the inner side of the tissue repair patch, while in yet further embodiments the tissue repair patch further comprises a biocompatible polymer layer on the outer side of the tissue repair patch. In further embodiments the structural component comprises electrocompacted collagen and chorion.

As used herein, the term “wound healing” refers to a regenerative process with the induction of an exact temporal and spatial healing program comprising wound closure and the processes involved in wound closure. The term “wound healing” encompasses but is not limited to the processes of granulation, neovascularization, fibroblast, endothelial and epithelial cell migration, extracellular matrix deposition, re-epithelialization, and remodeling.

In some embodiments, a method for “accelerating wound healing” is provided, whereby different aspects of the wound healing process are “enhanced.” As used herein, the term “enhanced” indicates that the methods provide an increased rate of wound healing. In preferred embodiments, the term “enhanced” indicates that the wound healing rate and/or a wound healing process occurs at least 10% faster than is observed in untreated or control-treated wounds. In particularly preferred embodiments, the term “enhanced” indicates that the wound healing rate and/or a wound healing process occurs at least 15% faster than is observed in untreated or control-treated wounds. In still further preferred embodiments, the term “enhanced” indicates that the wound healing rate and/or a wound healing process occurs at least 20% (e.g., 50%, 100%, etc.) faster than wounds untreated or control-treated wounds.

Contacting, as used herein, refers to causing two items to become physically adjacent and in contact, or placing them in an environment where such contact will occur within a reasonably short timeframe. For example, contacting a wound with a tissue repair patch includes positioning the tissue repair patch at or near a wound or defect such that the patch will interact with the wound or defect to stimulate enhanced wound healing.

In some embodiments, additional measures are taken to fix the tissue repair patch to the tissue wound or defect. In some embodiments, the tissue repair patch is adhered or stitched to the tissue wound or defect, typically through attachment to healthy tissue adjacent to the tissue wound or defect. In other embodiments, the tissue repair patch is fixed to the site using interpositional layering.

As used herein, the term “wound” refers to a disruption of the normal continuity of living structures (e.g., tissue and/or organs) caused by a physical (e.g., mechanical) force, a biological (e.g., thermic or actinic force), or a chemical means. The term “wound” also encompasses contused wounds, as well as incised, stab, lacerated, open, penetrating, puncture, abrasions, grazes, burns, frostbites, corrosions, wounds caused by ripping, scratching, pressure, and biting, and other types of wounds.

A tissue defect, as used herein, a weakness or other problem present in tissue. Tissue defects can occur from a variety of conditions that have stressed or damaged tissue, such as trauma, tumor, ulceration, or infection, or delivery in the case of vaginal tissue.

The tissue repair patch can be used to repair a wide variety of different types of wounds and/or defects in a wide variety of different types of tissue. In many embodiments, the tissue is soft tissue, which includes all tissues in the body that have not been hardened by ossification or calcification such as bones and teeth. Soft tissue includes muscle tissue, tendons, ligaments, fibrous tissue, skin, and various organs. In some embodiments, the tissue wound or defect is a genitourinary tissue wound or defect. In further embodiments, the tissue wound or defect is a bladder wound or defect. In yet further embodiments, the genitourinary tissue wound or defect is an obstetric-related vesico-vaginal fistula.

### Preparation of Structural and Structural/Regenerative Layers

The inventors have also developed special methods for preparing electrocompacted structural layers, including structural layers that have been combined with the regenerative layer (i.e., a structural/regenerative layer).

The inventors have developed a method of preparing electrocompacted amnio and collagen sheet for fistula repair. Placenta is collected, and amniotic membrane is physically isolated from the amniotic membrane by stripping the amniotic membrane of the placenta from the chorion layer. Amniotic membrane can be prepared for cryopreservation by, for example washing the amniotic membrane multiple (e.g., 3) times with saline solution (e.g., PBS) containing antibiotic (e.g., Pen/Strep/Amphotericin B) together with glycerol or DMSO. Freeze dried amniotic membrane can then chopped, milled, or otherwise broken into small pieces, which are then mixed with collagen. The mix of amnion and collagen can be lyophilized to form a sheet, which can then be electrocompacted to form a structural/regenerative layer including both amnion and collagen.

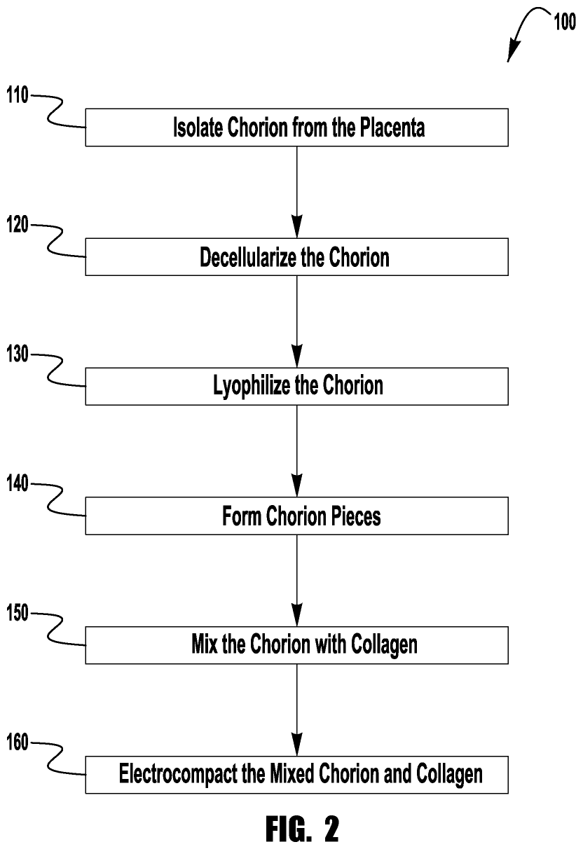

The inventors have also developed a method for preparing electrocompacted sheets including chorion and collagen 100. This process is shown in FIG. 2. The chorion layer of the placenta is first physically isolated from the placenta 110. The chorion is then decellularized using a detergent 120. The chorion is then lyophilized 130 and broken into small pieces (e.g., by cryomilling) 140. Dialyzed collagen is then mixed with the chorion pieces 150, and the co-electrocompacted 160 to form a structural layer including chorion and collagen. For example, collagen can be loaded into a window having the shape of the desired tissue repair patch within a plate, positioned over a cathode, chorion pieces can be added to the collagen, an anode positioned over the plate, and then current applied to provide an electrocompacted structural layer comprising chorion and collagen.

The present invention is illustrated by the following examples. It is to be understood that the particular examples, materials, amounts, and procedures are to be interpreted broadly in accordance with the scope and spirit of the invention as set forth herein.

### EXAMPLE

### In vitro Response of Human Buccal Epithelial Cells to a Multiphasic Bladder Patch

Replacing bladder tissue with a functional equivalent remains one of the most challenging problems for conditions such as iatrogenic injuries, trauma, or fistula in reconstructive urology. Conventional bladder reconstruction utilizing gastrointestinal (GIS) segments is the most frequently employed therapeutic strategy and is related to a series of complications due to incompatibility of GIS with urine. Various tissue-engineered scaffolds have been studied as bladder patches for bladder reconstruction. Synthetic polymer materials are generally preferred because they are relatively easy to manufacture, and biocompatibility has been studied in detail. Among the polymers examined for tissue engineering, polycaprolactone (PCL) exhibits ideal biocompatibility for bladder augmentation, and its degradation products have minimal toxicity in vivo. While PCL represents an excellent base material as a template for infiltrating tissue, it does not provide biochemical cues to guide cell differentiation. Utilization of naturally derived biomaterials such as amnion and collagen can provide biochemical cues for cell attachment, growth, and proliferation. Additionally, oral epithelial cells represent a viable source of cells for clinical applications as they can be collected from small biopsies and expanded for tissue regeneration.

### Methods:

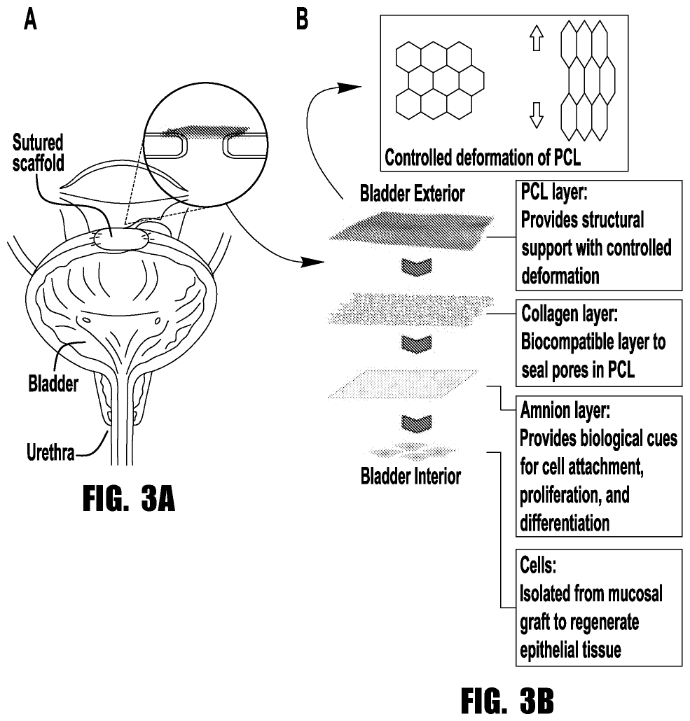

Human oral epithelial cells were harvested from buccal grafts under an IRB-approved protocol, and their phenotype was assessed by immunocytochemistry using a fluorophore-conjugated antibody against CK3/2p. Electrochemically compacted collagen sheets were fabricated using planar electrodes and then crosslinked using genipin. Younesi et al. Biofabrication, 7(3):035001 (2015). An example of where the tissue repair patch can be positioned and adhered using stiches for bladder repair is shown in FIG. 3A. The human amniotic membrane was isolated from the placenta and adhered to a genipin crosslinked collagen sheet/PCL using the gluing solution. A bladder repair patch having a polymer layer, a collagen layer, an amnion layer, and a layer of epithelial cells is shown in FIG. 3B. The morphology and composition of the surface layer were evaluated by scanning electron microscopy (SEM). PCL was laser cut using an Epilog laser system. The amnion section of bladder patch was seeded with human oral epithelial cells and cultured for three days following sterilization. Seeded collagen sheets were evaluated for proliferation using MTT assay. Western blot for CK3/2p was performed on cells seeded on bladder patch to determine their phenotype at 72 hours.

### Results:

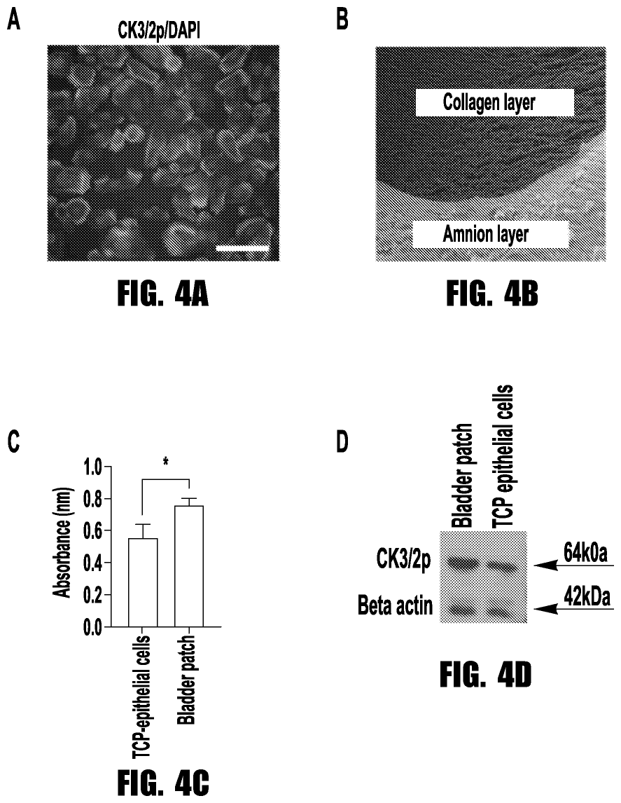

Human epithelial cells stained positive for CK3/2p (FIG. 4A). SEM showed a connection between layers within composite bladder patch (FIG. 4B). Epithelial cells exhibited increased cell proliferation when seeded on bladder patch compared to epithelial cells that are seeded on the culture plate (p<0.05) (FIG. 4C). Protein expression highlighted the presence of CK3/2p at 72 hours following seeding on a bladder patch (FIG. 4D).

### Conclusions:

Multiphasic bladder patch supports sufficient epithelial cell attachment and survival suitable for implantation. Protein expression results indicate that the multiphasic bladder patch sustains the epithelial cell phenotype and increases the proliferation rate. The bioengineered bladder patch is highly novel, can be utilized in conjunction with epithelial cells and has tremendous potential for regenerative medicine-based repair of bladder tissue.

The complete disclosure of all patents, patent applications, and publications, and electronically available materials cited herein are incorporated by reference. Any disagreement between material incorporated by reference and the specification is resolved in favor of the specification. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A tissue repair patch having an outer side and an inner side, comprising a structural component comprising collagen and/or chorion and regenerative component comprising amniotic tissue.

2. The tissue repair patch of claim 1, wherein the structural component and the regenerative component are in separate adjacent layers, with the structural component being on the outer side and the regenerative component being on the inner side.

3. The tissue repair patch of claim 2, wherein the structural component is adhered to the regenerative component using a glue comprising sodium hyaluronate, dextran, and bovine serum albumin.

4. The tissue repair patch of claim 1, wherein the structural component and the regenerative component are mixed together in a single layer.

5. The tissue repair patch of claim 1, further comprising epithelial cells seeded on the inner side of the tissue repair patch.

6. The tissue repair patch of claim 5, wherein the epithelial cells are oral or urogenital epithelial cells.

7. The tissue repair patch of claim 1, further comprising a biocompatible polymer layer on the outer side of the tissue repair patch.

8. The tissue repair patch of claim 7, wherein the polymer layer comprises polycaprolactone (PCL).

9. The tissue repair patch of claim 1, wherein the tissue repair patch has a modulus of elasticity (MPa) ranging from 0.1 MPa to 10 MPa.

10. The tissue repair patch of claim 1, wherein the tissue repair patch has a thickness ranging from 0.1 mm to 3 mm.

11. The tissue repair patch of claim 1, wherein the tissue repair patch has a size ranging from 4 cm² to 100 cm².

12. The tissue repair patch of claim 1, further comprising one or more growth factors on the inner and/or outer side of the tissue repair patch.

13. The tissue repair patch of claim 1, wherein the structural component comprises electrocompacted collagen and chorion.

14. A method of tissue repair, comprising
positioning a tissue repair patch on a tissue wound or defect of a subject, the tissue repair patch having an outer side and an inner side, comprising a structural component comprising collagen and/or chorion and regenerative component comprising amniotic tissue.

15. The method of claim 14, wherein the structural component and the regenerative component of the tissue repair patch are in separate adjacent layers, with the structural component being on the outer side and the regenerative component being on the inner side.

16. The method of claim 14, wherein the structural component and the regenerative component of the tissue repair patch are mixed together in a single layer.

17. The method of claim 14, wherein the tissue repair patch further comprising epithelial cells seeded on the inner side of the tissue repair patch.

18. The method of claim 14, wherein the tissue repair patch further comprises a biocompatible polymer layer on the outer side of the tissue repair patch.

19. The method of claim 14, wherein the structural component comprises electrocompacted collagen and chorion.

20. The method of claim 14, wherein the tissue wound or defect is a genitourinary tissue wound or defect.

21. The method of claim 14, wherein the tissue wound or defect is a bladder wound or defect.

22. The method of claim 20, wherein the genitourinary tissue wound or defect is an obstetric-related vesico-vaginal fistula.

23. The method of claim 14, wherein the tissue repair patch is adhered or stitched to the tissue wound or defect.
